(19) United States
(12) Patent Application Publication
(10) Pub. No.: US 2015/0032128 A1
(43) Pub. Date: Jan. 29, 2015

(54) ULTRASONIC SKIN TREATMENT DEVICE WITH HAIR REMOVAL CAPABILITY

(71) Applicant: SLENDER MEDICAL LTD., Herzliya (IL)

(72) Inventors: Doron-Aharon Tavlin, Minneapolis, MN (US); Yossi Gross, Moshav Mazor (IL); Haim Azhari, Doar-Na Misgav (IL); Efraim Cohen-Arazi, Herzliya (IL); Liat Tsoref, Tel Aviv (IL)

(21) Appl. No.: 14/374,384

(22) PCT Filed: Jan. 24, 2013
(86) PCT No.: PCT/IL2013/050071

§ 371 Date: Jul. 24, 2014

Related U.S. Application Data
(60) Provisional application No. 61/590,845, filed on Jan. 26, 2012.

(51) Int. Cl. A61N 7/02; B26B 21/48

(52) U.S. Cl. CPC A61N 7/02; B26B 21/48; A61B 2017/00752
USPC 606/131

(57) ABSTRACT

Apparatus (10) is provided, including a skin-application portion (30), configured to move across a skin surface (75) of a subject. At least one acoustic element (80) is coupled to the skin-application portion and configured to be placed in acoustic contact with the skin, and to apply ultrasound energy to the skin. Additionally, at least one hair removal element (20) is coupled to the skin-application portion and removes hair from the skin of the subject. Other applications are also described.

### CROSS-REFERENCES TO RELATED APPLICATIONS

The present application claims the priority of U.S. Provisional Application 61/590,845 to Tavlin et al., entitled, “Ultrasonic skin treatment with hair removal capability,” filed on Jan. 26, 2012, which is incorporated herein by reference.

### FIELD OF THE APPLICATION

The present invention relates generally to skin treatment and particularly to methods and apparatus for skin rejuvenation by application of ultrasound energy.

### BACKGROUND OF THE APPLICATION

Skin, the body's largest organ, is composed of multiple layers. The outer layer, epidermis, is divided into several sublayers. Beneath the epidermis lies the dermis skin layer, which is composed of two layers, the upper papillary layer and the lower reticular layer.

A major structural component of the dermis skin layer is collagen, a fibrous protein, which contributes to skin strength and elasticity. As such, collagen formation, and in contrast age-related collagen decline, leads to changes in mechanical properties of the skin, such as texture and resilience. Thermal treatment of the skin can lead to thermal shrinkage of collagen, which occurs by the dissociation of heat-sensitive bonds of the collagen molecule. Thermal denaturing or shrinkage of collagen typically results in a tightening effect of the skin.

Visible effects of aging or damage of the skin are disturbing to many individuals and therefore methods for rejuvenation of maturing or damaged skin are of interest. Some skin rejuvenation methods include application of energy to heat selected areas of the skin in order to obtain an improvement in the appearance of the treated skin.

Removal of unwanted facial and body hair from the skin is considered by some to improve the appearance of skin.

### SUMMARY OF APPLICATIONS

Some applications of the present invention comprise apparatus and methods for skin treatment by providing a single apparatus for hair removal and for skin rejuvenation by application of energy thereto. As provided by some applications of the present invention, apparatus comprising a skin-application portion is moved across skin of a subject. Typically, at least one acoustic element, e.g., an ultrasound transducer, is coupled to the skin-application portion and is placed in acoustic contact with the skin of the subject. The acoustic element applies ultrasound energy, e.g., high intensity focused ultrasound (HIFU) energy, to the skin. Application of ultrasound energy to the skin surface with consequent heating of underlying skin layers typically leads to collagen shrinkage and remodeling, resulting in skin tightening and rejuvenation. Additionally, at least one hair removal element, e.g., a razor blade and/or an epilation element, is coupled to the skin-application portion and removes hair from the skin of the subject when the skin-application portion is moved across skin of a subject.

For some applications, a substance for lubricating the passage of the hair removal element over the skin, e.g., a gel, a cream and/or a foam, is applied to a portion of the skin surface designated for treatment. Typically, the lubricating substance additionally serves as an acoustic coupling medium between the ultrasound transducer and the skin surface. For such applications, the ultrasound transducer is placed in acoustic contact with the skin and is activated to apply ultrasound energy to the skin while the skin-application portion is moved across the skin. Subsequently, as the skin-application portion is moved across the skin, the hair removal element which is coupled to the skin-application portion removes the lubricating substance along with hair of the subject.

There is therefore provided, in accordance with some applications of the present invention, apparatus, including:

a skin-application portion, configured to move across skin of a subject;

at least one acoustic element coupled to the skin-application portion and configured to be placed in acoustic contact with the skin, and configured to apply ultrasound energy to the skin; and

at least one hair removal element coupled to the skin-application portion and configured to remove hair from the skin of the subject.

For some applications the apparatus includes a handle, and the at least one acoustic element is disposed between the hair removal element and the handle.

For some applications, the at least one hair removal element is configured to remove hair from the skin of the subject subsequently to application of the ultrasound energy to the skin by the acoustic element.

For some applications, the at least one hair removal element is configured to remove hair from the skin of the subject subsequently to application of the ultrasound energy to the skin by the acoustic element.

For some applications the apparatus includes a control unit which is configured to limit application of energy to the skin, after a skin treatment, during a time period that includes a time that is greater than 1 hour and less than 10 days following the skin treatment.

For some applications, the control unit is configured to limit the application of the energy to the skin during a time period that includes a time that is greater than 18 hours and less than 7 days following the skin treatment.

For some applications the apparatus includes:

circuitry configured to generate a current responsive to motion of the skin-application portion; and

a control unit, which is configured to receive the current, to determine, responsive thereto, whether the skin-application portion is moving with respect to the skin, and to drive the acoustic element to apply the ultrasound energy to the skin responsive to determining that the skin-application portion is moving with respect to the skin.

For some applications the apparatus includes a pressure sensor configured to generate a pressure sensor signal responsive to contact of the skin-application portion with the skin, and the control unit is adapted to receive the pressure sensor signal, to determine, responsive thereto, a degree of contact of the skin-application portion with the skin, and to control application of ultrasound energy from the acoustic element to the skin responsive to determining the contact of the skin-application portion with the skin.

For some applications the apparatus includes:

a sensor configured to generate a sensor signal, the sensor selected from the group consisting of: an optical sensor and an electromagnetic sensor; and

a control unit, which is configured to drive the acoustic element to apply the ultrasound energy to the skin responsive to the sensor signal.

For some applications, the apparatus is configured to sense a temperature of the skin and to regulate the application of the ultrasound energy in response thereto.

For some applications, the acoustic element includes an ultrasound transducer configured to apply the ultrasound energy as high intensity focused ultrasound (HIFU) energy.

For some applications, the acoustic element includes a piezoelectric element.

For some applications, the hair removal element includes a blade.

For some applications, the skin-application portion is configured for use with a lubricating substance for providing acoustic coupling between the acoustic element and the skin of the subject and for facilitating hair removal by the blade, and the blade is configured to remove the substance from the skin following application of the ultrasound energy to the skin, during removal of hair from the skin.

For some applications, the hair removal element includes a non-blade epilation element.

For some applications, the skin-application portion is configured for use with a lubricating substance for providing acoustic coupling between the acoustic element and the skin of the subject, and the skin-application portion further includes a protruding surface which is configured to remove the substance from the skin following application of the ultrasound energy to the skin, prior to removal of hair from the skin by the non-blade epilation element.

For some applications the apparatus includes a user-controllable control element, configured to control a depth of a focal zone of the acoustic element.

There is additionally provided, in accordance with some applications of the present invention, a method, including:

providing an acoustic element and a hair removal element, both coupled to a skin-application portion of an apparatus;

activating the acoustic element to apply ultrasound energy to skin of a subject; and

subsequently, removing hair from the skin of the subject using the hair removal element, while the skin-application portion is moving across the skin.

For some applications, activating the acoustic element includes moving the acoustic element across the skin, while the acoustic element is in acoustic contact with the skin.

For some applications, activating the acoustic element includes placing the acoustic element in contact with the skin.

For some applications the method includes:

applying a lubricating substance to the skin for providing acoustic coupling between the acoustic element and the skin of the subject and for facilitating hair removal;

subsequently, performing the activating of the acoustic element; and

subsequently, using a blade, performing the removing of the hair while removing the lubricating substance using the blade.

For some applications, providing includes providing apparatus that is configured to:

generate a current responsive to motion of the acoustic element across the skin;

determine, responsive to the current, whether the acoustic element is moving with respect to the skin; and

control application of ultrasound energy from the acoustic element to the skin responsive to determining that the acoustic element is moving with respect to the skin.

There is also provided, in accordance with some applications of the present invention, apparatus, including:

a skin-application portion, configured to move across skin of a subject;

at least one energy source coupled to the skin-application portion and configured to be placed in contact with the skin, and configured to apply energy to the skin; and

at least one hair removal element coupled to the skin-application portion and configured to remove hair from the skin of the subject.

For some applications, the energy source includes an energy source selected from the group consisting of: a microwave energy source, a radio frequency energy source, an optical energy source and an electrical energy source.

The present invention will be more fully understood from the following detailed description of applications thereof, taken together with the drawings, in which:

### BRIEF DESCRIPTION OF THE DRAWINGS

FIG. 1 is a schematic illustration of apparatus for skin treatment, in accordance with some applications of the present invention;

FIGS. 2A-B are schematic illustrations of a cross section of the apparatus for skin treatment, in accordance with some applications of the present invention;

FIG. 3 is a schematic illustration of apparatus for skin treatment, in accordance with some applications of the present invention; and

FIG. 4 is a schematic illustration of apparatus for skin treatment positioned in contact with skin surface of a subject, in accordance with some applications of the present invention.

### DETAILED DESCRIPTION OF APPLICATIONS

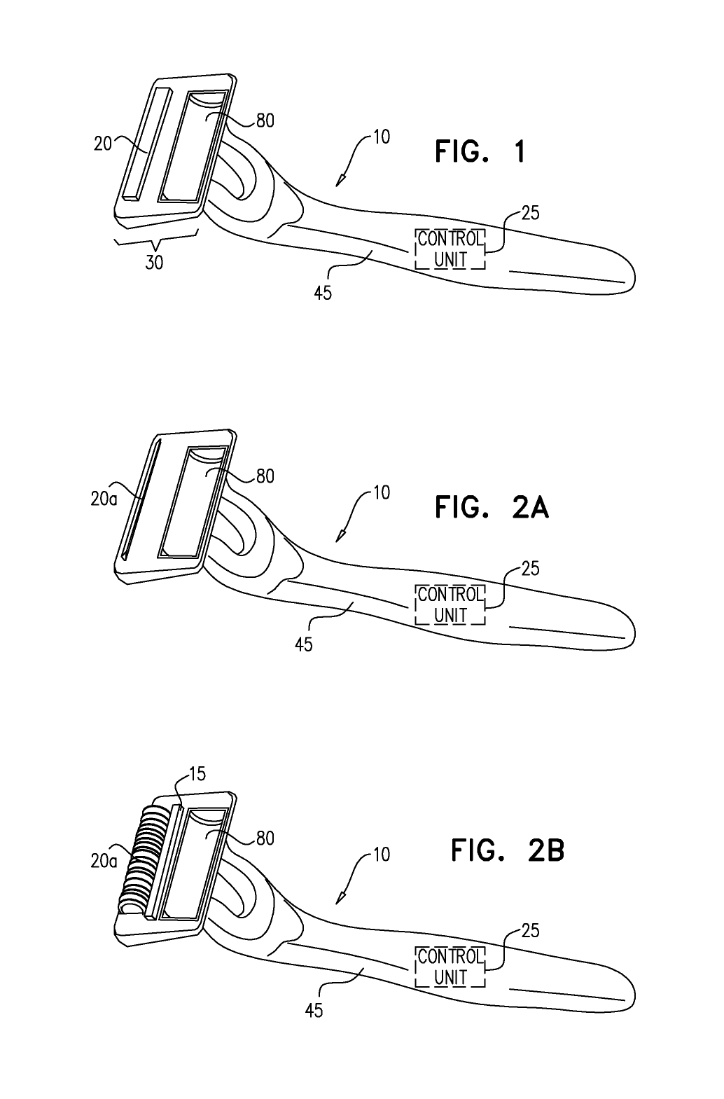

Reference is made to FIGS. 1 and 2A-B, which are schematic illustrations of apparatus 10 for dual skin treatment, in accordance with some applications of the present invention. Apparatus 10 typically comprises handheld apparatus 10 comprising a skin-application portion 30 that is moved across a selected region of skin of a subject, e.g., skin on a subject's face, arms, or legs.

Typically, skin application portion 30 comprises at least one acoustic element, e.g., an ultrasound transducer 80, that is placed in acoustic contact with the skin of the subject. Transducer 80 applies ultrasound energy, e.g., high intensity focused ultrasound (HIFU) energy, to the skin. Application of ultrasound energy to the skin surface with subsequent heating of underlying skin layers typically leads to collagen shrinkage and remodeling, resulting in tightening of the skin, wrinkle reduction and a rejuvenated appearance of the skin. Transducer 80 is shown as a semi-cylindrical transducer by way of illustration and not limitation. It is noted that other configurations of transducer 80 may be used, including, but not limited to, a spherical-shaped transducer. Typically, ultrasound transducer 80 comprises a piezoelectric element.

Skin-application portion 30 additionally comprises a hair removal element 20, e.g., a razor blade 20a and/or a spring epilator 20b (as shown in FIGS. 2A-B). Hair removal element 20 removes hair on a skin surface of the subject when skin-application portion 30 is moved across the skin. It is noted that other epilation elements and techniques may be used.

Typically, apparatus 10 further comprises a handle 45. Transducer 80 is disposed between hair removal element 20 and handle 45. Such a configuration of apparatus 10 facilitates application of ultrasound energy to the skin followed by hair removal by element 20 as skin-application portion 30 is moved across the skin.

Typically, razor blade 20a removes a lubricating substance (shown in FIG. 4) which is applied to the skin prior to the treatment, and also acts as an acoustic coupling medium between the ultrasound transducer and the skin surface. For applications in which hair removal element 20 comprises a non-blade epilator, such as spring epilator 20b, skin-application portion 30 comprises a surface 15 for removal of the lubricating substance from the skin prior to hair removal by the epilation element.

Skin-application portion 30 is typically but not necessarily disposable and/or sterile.

Skin-application portion 30 typically comprises circuitry which generates a current responsive to motion of skin-application portion 30 across the skin. A control unit 25 receives the current from the circuitry and determines, in response thereto, whether skin-application portion 30 is moving with respect to the skin, and drives transducer 80 to apply the ultrasound energy to the skin while skin-application portion 30 is moving with respect to the skin.

Additionally or alternatively, the circuitry generates a current responsive to contact of skin-application portion 30 with the skin. Control unit 25 receives the current from the circuitry and determines, in response thereto, whether skin-application portion 30 is in contact with the skin, and drives transducer 80 to apply the ultrasound energy to the skin while skin-application portion 30 is in contact with respect to the skin.

Typically, control unit 25 is also configured to alter application of the energy from transducer 80, when motion of skin-application portion 30 across the skin is altered. For example, application of energy from transducer 80 may be discontinued or reduced when skin-application portion 30 is moving slowly or is not moving across the skin of the subject, in order to prevent excessive heating of the skin.

For some applications, control unit 25 is programmed to alter application of the energy from transducer 80 during predetermined and/or selected time periods. For example, apparatus 10 may be configured to limit continuous application of energy from transducer 80 in order to prevent excessive heating of the skin. Additionally or alternatively, control unit 25 limits activation of ultrasound transducer 80 to daily, once every few days, once a week, or some other usage frequency, in order to allow recovery of the skin and underlying collagen matrix. For example, control unit 25 limits application of energy to the skin, after a skin treatment, during a time period that includes a time that is greater than 1 hour and less than 10 days following the skin treatment, such as during a time period that is greater than 18 hours and less than 7 days following the skin treatment. Apparatus 10 continues to function as a device for daily hair removal even when activation of ultrasound transducer 80 is limited by control unit 25.

For some applications, transducer 80 includes a fluid that is disposed between the piezoelectric element within transducer 80 and a membrane that is located on the outer surface of transducer 80. Transducer 80 is typically placed on the skin surface of the subject such that the membrane is in contact with the skin. The fluid typically applies pressure to the membrane, causing inflation and protruding of the membrane, which results in improved contact between the membrane and the skin. Typically, the fluid is at room temperature, e.g., 20-25° C. Alternatively the fluid is cooled, e.g., to 5-15° C. and used for cooling by removing excess heat from the transducer and from the outer layer of the skin.

It is noted that application of ultrasound energy to the skin causes heating and consequent damage to blood vessels which supply hair follicles within the skin. Thus, re-growth of hair may be inhibited, reducing the need for repeated hair removal treatments, at the same time that a hair removal treatment is applied.

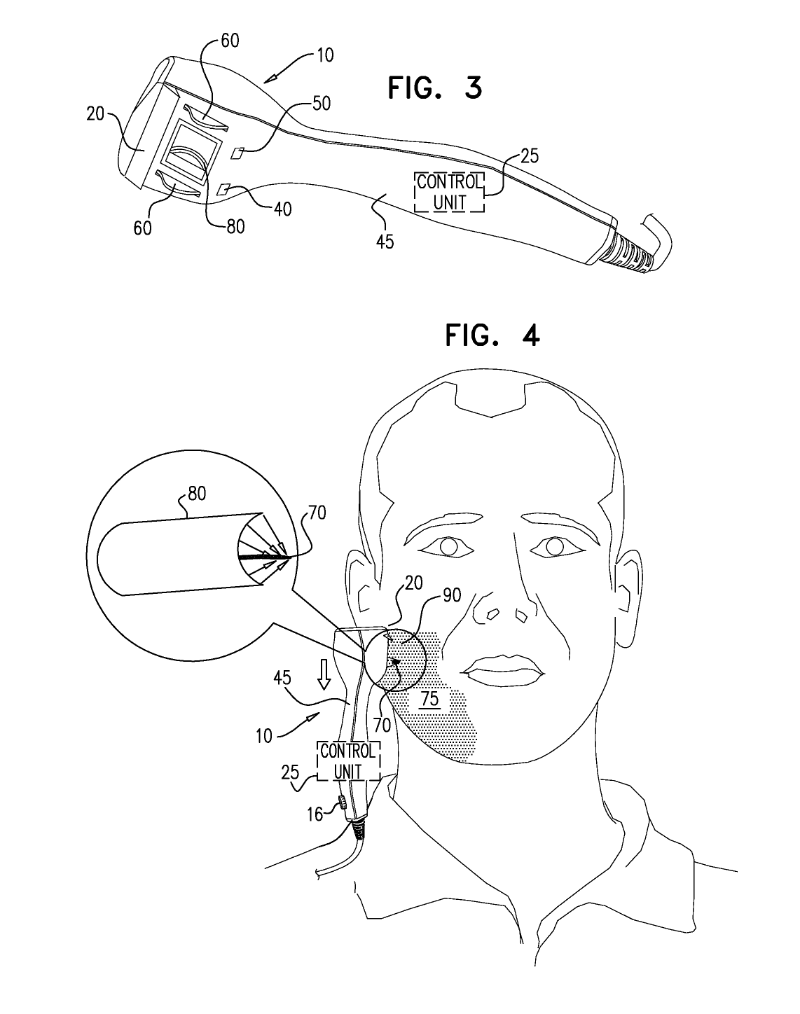

Reference is made to FIG. 3, which is a schematic illustration of apparatus 10 for skin treatment, in accordance with some applications of the present invention.

For some applications, apparatus 10 comprises one or more electromechanical sensors, e.g., pressure sensor 60, which generate a pressure sensor signal responsive to contact of skin-application portion 30 with the skin. Typically, control unit 25 receives the pressure sensor signal and determines, responsive thereto, a degree of physical contact of skin-application portion 30 with the skin, and controls application of ultrasound energy from the transducer 80 to the skin responsive to determining the contact of the skin-application portion with the skin. Accordingly, control unit 25 is configured to alter application of the energy from transducer 80, when contact of skin-application portion 30 with the skin is altered. For example, application of energy from transducer 80 may be discontinued when skin-application portion 30 is sensed by pressure senor 60 as not being in sufficient contact with the skin of the subject.

Additionally or alternatively, apparatus 10 comprises other sensing modalities, e.g., an optical sensor 50 (like that used in a computer mouse) and/or an electromagnetic sensor 40 (like that used in a computer mouse) or a combination thereof. For some applications, apparatus 10 comprises sensors for measuring various parameters, e.g., applied pressure on the skin, location of apparatus 10 on the skin, temperature of the apparatus or skin, and motion of the apparatus with respect to the skin.

Reference is made to FIG. 4, which is a schematic illustration of apparatus 10 positioned in contact with a skin surface 75, in accordance with some applications of the present invention. Transducer 80 typically focuses energy transmission to a particular area of the skin in order to enable treatment of a small focal zone 70. FIG. 4 shows an exploded view of semi-cylindrical transducer 80 transmitting ultrasound energy toward focal zone 70. Typically, the ultrasound energy emitted from transducer 80 is focused such that focal zone 70 is disposed beneath the outer layer of the skin, the epidermis, so as to avoid damage to the epidermis and to trigger collagen shrinkage at the focal zone. Focal zone 70 is typically located at least 0.25 mm beneath the outer layer of the skin, or less than 2 mm beneath the outer layer of the skin, or between 0.25 and 2 mm beneath the outer layer of the skin. Typically, the semi-cylindrical configuration of transducer 80 creates a linear shaped focal zone 70 as shown in FIG. 4. As mentioned hereinabove, transducer 80 is shown as a semi-cylindrical transducer by way of illustration and not limitation. It is noted that other configurations of transducer 80 may be used, including, but not limited to, a spherical-shaped transducer. Typically, ultrasound transducer 80 comprises a piezoelectric element.

Typically, apparatus 10 applies focused energy to more than one depth beneath skin surface 75, in order to facilitate several aspects of skin treatment. For example, focused energy transmission to a focal zone 70 that is less than 0.2 mm from skin surface 75 facilitates delivery of large facial cream molecules which are applied to the skin surface prior to treatment. Typically, the facial cream comprises a therapeutic and/or cosmetic agent. Additionally or alternatively, apparatus 10 applies focused energy to a focal zone 70 that is located 0.2-1 mm beneath the outer layer of the skin, in order to enhance collagen shrinkage and wrinkle reduction. Further additionally or alternatively, apparatus 10 applies focused energy to a focal zone 70 that is located deeper than 1 mm from the skin surface in order to affect hair follicles and inhibit re-growth of hair. For some such applications, apparatus 10 comprises a user-controllable control element 16 (e.g., a dial, switch, or button) coupled to apparatus 10 and configured to control a depth of focal zone 70 and allow changing the depth of a focal zone typically in response to user input. Alternatively or additionally, apparatus 10 automatically varies the focal depth among two or more of the above depth ranges.

Typically, a lubricating substance 90, e.g., a gel, a cream and/or a foam, is applied to skin surface 75 which is designated for treatment. Typically, lubricating substance 90 serves as an acoustic coupling medium between ultrasound transducer 80 and skin surface 75. Additionally, lubricating substance 90 serves as a lubricant in order to facilitate passage of hair removal element 20 over skin surface 75, similar to shaving cream. For such applications, ultrasound transducer 80 is placed in acoustic contact with the skin and is activated to apply ultrasound energy to the skin while the skin-application portion is moved across the skin. Subsequently, as the skin-application portion is moved across the skin (as indicated by the arrow in FIG. 4), hair removal element 20 which is coupled to the skin-application portion removes lubricating substance 90 along with hair of the subject. As described hereinabove with reference to FIG. 2B, for applications in which hair removal element 20 comprises a non-blade epilator, such as spring epilator 20b, skin-application portion 30 comprises a protruding surface 15 (or other mechanism, e.g., suction) for removal of substance 90 from the skin prior to hair removal by epilation element 20b. For some applications, following removal of lubricating substance 90 by hair removal element 20 or surface 15, further effects of ultrasound transducer 80 on the skin are minimized during that treatment, and the skin is ready for a follow up treatment by ultrasound transducer 80 the next time (e.g., the next day) that lubricating substance 90 is applied.

It is noted that, lubricating substance 90 is typically degassed such that a minimal number of gas bubbles are present in substance 90, in order to avoid blocking or attenuation of the ultrasonic waves.

For some applications, apparatus 10 is configured to vibrate hair shafts of the subject. For such applications, apparatus 10 comprises an additional acoustic element that generates a lower frequency signal (e.g., at least 1 kHz, less than 2 MHz and/or between 1 kHz-2 MHz) which causes vibration of the hair shafts. Alternatively, transducer 80 applies high intensity focused ultrasound (HIFU) energy in short bursts of frequency (e.g., 1-100 kHz), such that the bursts of energy create mechanical pressure that vibrate the hair shafts. Typically, vibration of hair shafts by apparatus 10 as described enhances the hair removal functionality of apparatus 10.

Reference is made to FIGS. 1-4. It is to be noted that apparatus 10 for skin treatment may comprise an alternative energy source which applies energy to the skin, e.g., a microwave energy source, and/or a radio frequency energy source, and/or optical energy source (for example, an infra-red and/or a visible light source) and/or an electrical energy source or a combination thereof. Additionally or alternatively, any of these energy sources may be used in combination with ultrasound transducer 80.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. Apparatus, comprising:
a skin-application portion, configured to move across skin of a subject;
at least one acoustic element coupled to the skin-application portion and configured to be placed in acoustic contact with the skin, and configured to apply ultrasound energy to the skin;
at least one hair removal element coupled to the skin-application portion and configured to remove hair from the skin of the subject; and
a handle,
wherein the at least one acoustic element is disposed between the hair removal element and the handle.

2. (canceled)

3. (canceled)

4. The apparatus according to claim 1, wherein the at least one hair removal element is configured to remove hair from the skin of the subject subsequently to application of the ultrasound energy to the skin by the acoustic element.

5. The apparatus according to claim 1, further comprising a control unit which is configured to limit application of energy to the skin, after a skin treatment, during a time period that includes a time that is greater than 1 hour and less than 10 days following the skin treatment.

6. The apparatus according to claim 5, wherein the control unit is configured to limit the application of the energy to the skin during a time period that includes a time that is greater than 18 hours and less than 7 days following the skin treatment.

7. The apparatus according to claim 1, further comprising:
circuitry configured to generate a current responsive to motion of the skin-application portion; and
a control unit, which is configured to receive the current, to determine, responsive thereto, whether the skin-application portion is moving with respect to the skin, and to drive the acoustic element to apply the ultrasound energy to the skin responsive to determining that the skin-application portion is moving with respect to the skin.

8. The apparatus according to claim 7, further comprising a pressure sensor configured to generate a pressure sensor signal responsive to contact of the skin-application portion with the skin, and wherein the control unit is adapted to receive the pressure sensor signal, to determine, responsive thereto, a degree of contact of the skin-application portion with the skin, and to control application of ultrasound energy from the acoustic element to the skin responsive to determining the contact of the skin-application portion with the skin.

9. The apparatus according to claim 1, further comprising:
a sensor configured to generate a sensor signal, the sensor selected from the group consisting of: an optical sensor and an electromagnetic sensor; and
a control unit, which is configured to drive the acoustic element to apply the ultrasound energy to the skin responsive to the sensor signal.

10. The apparatus according claim 1, wherein the apparatus is configured to sense a temperature of the skin and to regulate the application of the ultrasound energy in response thereto.

11. The apparatus according to claim 1, wherein the acoustic element comprises an ultrasound transducer configured to apply the ultrasound energy as high intensity focused ultrasound (HIFU) energy.

12. The apparatus according to claim 1, wherein the acoustic element comprises a piezoelectric element.

13. The apparatus according to claim 1, wherein the hair removal element comprises a blade.

14. The apparatus according to claim 13, wherein the skin-application portion is configured for use with a lubricating substance for providing acoustic coupling between the acoustic element and the skin of the subject and for facilitating hair removal by the blade, and wherein the blade is configured to remove the substance from the skin following application of the ultrasound energy to the skin, during removal of hair from the skin.

15. The apparatus according to claim 1, wherein the hair removal element comprises a non-blade epilation element.

16. The apparatus according to claim 15, wherein the skin-application portion is configured for use with a lubricating substance for providing acoustic coupling between the acoustic element and the skin of the subject, and wherein the skin-application portion further comprises a protruding surface which is configured to remove the substance from the skin following application of the ultrasound energy to the skin, prior to removal of hair from the skin by the non-blade epilation element.

17. The apparatus according to claim 1, further comprising a user-controllable control element, configured to control a depth of a focal zone of the acoustic element.

18. A method comprising:
providing an acoustic element and a hair removal element, both coupled to a skin-application portion of an apparatus;
activating the acoustic element to apply ultrasound energy to skin of a subject; and
subsequently, removing hair from the skin of the subject using the hair removal element, while the skin-application portion is moving across the skin.

19. The method according to claim 18, wherein activating the acoustic element comprises moving the acoustic element across the skin, while the acoustic element is in acoustic contact with the skin.

20. The method according to claim 18, wherein activating the acoustic element comprises placing the acoustic element in contact with the skin.

21. The method according to claim 18, further comprising:
applying a lubricating substance to the skin for providing acoustic coupling between the acoustic element and the skin of the subject and for facilitating hair removal;
subsequently, performing the activating of the acoustic element; and
subsequently, using a blade, performing the removing of the hair while removing the lubricating substance using the blade.

22. The method according to claim 18, wherein providing comprises providing apparatus that is configured to:
generate a current responsive to motion of the acoustic element across the skin;
determine, responsive to the current, whether the acoustic element is moving with respect to the skin; and
control application of ultrasound energy from the acoustic element to the skin responsive to determining that the acoustic element is moving with respect to the skin.

23. (canceled)

24. (canceled)
